(19) United States
(12) Patent Application Publication
(10) Pub. No.: US 2011/0098265 A1
(43) Pub. Date: Apr. 28, 2011

(54) METHODS FOR REDUCING CRAVINGS AND IMPULSES ASSOCIATED WITH ADDICTIVE AND COMPULSIVE BEHAVIORS

(75) Inventors: Michael J. Bull, Somerset, WI (US); Gottfried H. Kellermann, Osceola, WI (US); Kelly L. Olson, St. Croix Falls, WI (US)

(73) Assignee: NEUROSCIENCE, INC., Osceola, WI (US)

(21) Appl. No.: 12/913,860

(22) Filed: Oct. 28, 2010

Related U.S. Application Data
(60) Provisional application No. 61/255,608, filed on Oct. 28, 2009.

(51) Int. Cl. A61K 31/555; A61K 31/4748; A61P 25/00; A61P 25/30; A61P 3/04

(52) U.S. Cl. 514/188; 514/295

(57) ABSTRACT

This document provides methods and materials related to managing weight, supporting appetite control, and controlling cravings associated with smoking reduction or cessation regimens and/or nicotine reduction or cessation regimens. For example, compositions comprising an agent to support acetylcholine and an agent to support one or more biogenic amines, and methods for using such compositions for craving and appetite control are provided. Methods and materials to reduce cravings associated with the reduction or cessation of the use of chemical substances (e.g., drugs of abuse, including opioids, cocaine, methamphetamine, cannabis, alcohol), and to reduce cravings associated with addictive and/or compulsive behaviors (e.g., gambling, sex, and repetitive behaviors) are also provided.

### CROSS-REFERENCE

This application claims the benefit of U.S. Provisional Application No. 61/255,608, filed Oct. 28, 2009, which application is incorporated herein by reference.

### BACKGROUND

1. Technical Field

This document provides methods and materials related to reducing cravings and impulses associated with addictive and compulsive behaviors. For example, this document provides compositions comprising an agent to support acetylcholine and an agent to support one or more biogenic amines, and methods for using such compositions for controlling cravings associated with behavior modification programs, addictive and/or compulsive behaviors (e.g., gambling, sex, and repetitive behaviors), or the reduction or cessation of the use of chemical substances (e.g., nicotine, tobacco, drugs of abuse, including stimulants, narcotics, depressants, cannabis, hallucinogens, inhalants, steroids, and alcohol). The methods and materials provided herein may also be used to manage weight, support appetite control, and control cravings associated with smoking reduction or cessation regimens and/or nicotine reduction or cessation regimens.

2. Background Information

In spite of the immediate and long-term health benefits of quitting addictive or compulsive behaviors such as a smoking, drug abuse, gambling and sex addictions, and obsessive-compulsive disorder, persistent cravings or urges associated with efforts to modify one's behavior can discourage many individuals from taking steps to treat an addiction. For example, weight gain is a common side-effect associated with reduction or cessation of smoking. The prospect of weight gain is enough to discourage many smokers from taking steps to treat or reduce nicotine addiction. Nicotine is an appetite suppressant and metabolic stimulant and, therefore, former smokers typically experience increased food cravings, appetite, and calorie intake. Weight gain associated with smoking cessation may be due, at least in part, to decreased activity of the adrenergic nervous system. Decreased adrenergic nervous system activity is associated with decreased mobilization and oxidation of fatty acids and, consequently, increased fat storage. Smoking cessation products themselves also can contribute to weight gain. In addition to food cravings, persistent cravings for nicotine make compliance with smoking or nicotine reduction programs difficult.

There is a need for more effective methods of reducing cravings and impulses associated with addictive behaviors such as a smoking or using other chemical substances. Methods of reducing such cravings would also promote preventing or reducing weight gain commonly associated with smoking cessation and/or nicotine replacement therapies, particularly methods that reduce cravings, enhancing feelings of satiety, and, thereby, increase patient compliance and satisfaction with smoking cessation programs.

Studies have demonstrated the importance of maintaining neurotransmitter balance for curbing craving- and addiction-related behaviors, particularly behaviors that effect appetite control, weight management, and compulsive and addictive impulses. In particular, imbalance in levels of the neurotransmitters acetylcholine, serotonin, dopamine, gamma-aminobutyric acid, glutamate, and norepinephrine is associated with changes in cravings, appetite, and weight gain. A variety of compounds have different activities related to preventing or correcting neurotransmitter imbalance. For example, some botanical extracts have been associated with neurotransmitter balance and weight loss. The neurotransmitter acetylcholine is released from cholinergic neurons and is required for the normal conduction of nerve impulses. Acetylcholine deficits, which can be caused by decreased acetylcholine synthesis and/or increased degradation, are associated with memory decline and reduced cognitive capacity. By blocking acetylcholine degradation, acetylcholinesterase inhibitors boost serum levels of acetylcholine. Huperzine A (HupA) is a potent and reversible inhibitor of acetylcholinesterase derived from the Chinese plant Huperzia serrata. HupA has been found to improve cognitive deficits and reduce cravings, possibly by increasing acetylcholine engagement of the nicotinic receptor. As compared to other acetylcholinesterase inhibitors, HupA has improved penetration through the blood-brain barrier, higher oral bioavailability, and longer duration of acetylcholinesterase inhibitory action. See Wang et al., Acta Pharmacol Sin. 27(1):1-26 (2006); Hanin et al., Ann N Y Acad. Sci. 695:304-306 (1993).

Other compounds associated with neurotransmitter balance include the botanical extracts fava and forskolin. Fava (from Vicia faba) is an abundant source of the dopamine precursor, levodopa. Levodopa (L-DOPA), which is used to treat symptoms associated with Parkinson's disease, correlates with weight loss frequently experienced by Parkinson's patients. Administering fava results in increased serum levels of 3,4-hydroxy-phenylacetic acid (DOPAC) and norepinephrine in addition to levodopa. Forskolin is the main active ingredient in the Ayurvedic herb Coleus forskohlii. Forskolin has been extensively researched in the medical field for use in the treatment of allergies, respiratory problems, cardiovascular diseases, glaucoma, psoriasis, and hypothyroidism. Forskolin increases cyclic AMP and appears to have additional actions that are due to its ability to alter a number of membrane transport proteins. Decreased adenylate cyclase transmembrane signaling activity has been associated with the development and maintenance of obesity.

### SUMMARY

This document provides methods and materials related to reducing cravings and impulses associated with addictive and compulsive behaviors. For example, provided herein are compositions or kits containing a combination of an agent to support acetylcholine and an agent to support biogenic amines, and methods for using a composition or kit as described herein for controlling cravings associated with behavior modification programs, addictive and/or compulsive behaviors (e.g., gambling, sex, and repetitive behaviors), or the reduction or cessation of the use of chemical substances (e.g., nicotine, tobacco, drugs of abuse, including opioids, cocaine, methamphetamine, and alcohol). In some cases, a composition or kit as described herein can be administered to a human to manage weight, prevent or reduce weight gain, support appetite control, and control cravings associated with smoking reduction or cessation regimens, nicotine reduction or cessation regimens, nicotine withdrawal, or nicotine cravings, or consuming smoking cessation medicaments. The compositions and kits herein can be useful to support appetite control, reduce total body weight and body mass index, reduce body fat, increase lean body mass, and reduce perceived appetite level. Such compositions can have substantial value for clinical use.

In general, this document features a composition. The composition can comprise, or consist essentially of, a therapeutically effective amount of an agent to support one or more amines.

In another aspect, this document features a composition. The composition can comprise, or consist essentially of, a therapeutically effective amount of an agent to support acetylcholine, an agent to support one or more biogenic amines, and an agent to support second messenger signal transduction. The composition can further comprise an agent to support second messenger signal transduction. The composition can further comprise a therapeutically effective amount of a decarboxylase inhibitor. The composition can further comprise one or more of chromium polynicotinate, L-glutamine, folic acid, pantothenic acid, vitamin C, vitamin B6, or L-phenylalanine, or pharmaceutically acceptable salts thereof, or any combination thereof.

In another aspect, this document features a composition. The composition can comprise, or consist essentially of, a therapeutically effective amount of an agent to support acetylcholine, e.g., Huperazine A in an amount between about 160 μg and about 240 μg, and an agent to support one or more biogenic amines in an amount between about 5 mg and about 150 mg. The composition can further comprising an agent to support second messenger signal transduction in an amount between about 0.5 mg and about 15 mg. The agent to support acetylcholine can be selected from the group consisting of Huperzine A, alpha glycerylphosphorylcholine, physostigmine, neostigmine, pyridostigmine, ambenonium, demarcarium, rivastigmine, phenanthrene derivatives, galantamine, donepezil, tacrine, edrophonium, donepezil, and diisopropyl phosphorofluoridate, or pharmaceutically acceptable salts thereof. The agent to support one or more biogenic amines can be selected from the group consisting of a neurotransmitter precursor, an amino acid or an amino acid derivative, and EGCG, or pharmaceutically acceptable salts thereof. The agent to support second messenger signal transduction can be selected from the group consisting of forskolin, sclareolide, a benzyloxybenzaldehyde analog, tauroursodeoxycholic acid, and tetrabutylammonium, or pharmaceutically acceptable salts thereof. The agent to support one or more biogenic amines can be selected from the group consisting of 5-hydroxytryptophan (5-HTP), EGCG, L-DOPA, N-acetyl-cysteine, cysteine, N-acetyl-tyrosine, tyrosine, D,L-phenylalanine, L-phenylalanine, L-histidine, L-theanine, L-tryptophan, or pharmaceutically acceptable salts thereof.

The composition can comprise Huperzine A, forskolin, 5-HTP, N-acetyl-cysteine, L-DOPA, and EGCG, or pharmaceutically acceptable salts thereof. The composition can comprise Huperzine A or a pharmaceutically acceptable salt thereof in a range of about 20 μg and about 5000 μg, 5-HTP or a pharmaceutically acceptable salt thereof in a range of about 5 mg and about 10,000 mg, N-acetyl-cysteine or a pharmaceutically acceptable salt thereof in a range of about 100 mg and about 100,000 mg, L-DOPA or a pharmaceutically acceptable salt thereof in a range of about 10 mg to about 20,000 mg, and EGCG or a pharmaceutically acceptable salt thereof in a range of about 10 mg and about 10,000 mg. The agent to support acetylcholine can comprise Huperzine A, or a pharmaceutically acceptable salt thereof. The therapeutically effective amount of Huperzine A or a pharmaceutically acceptable salt thereof can be between about 20 μg and about 5000 μg. The therapeutically effective amount of Huperzine A or a pharmaceutically acceptable salt thereof is between about 160 μg and about 240 μg. The therapeutically effective amount of Huperzine A or a pharmaceutically acceptable salt thereof can be about 240 μg. The agent to support second messenger signal transduction can comprise forskolin, or a pharmaceutically acceptable salt thereof. The therapeutically effective amount of forskolin or a pharmaceutically acceptable salt thereof can be between about 0.5 mg and about 2,000 mg. The therapeutically effective amount of forskolin or a pharmaceutically acceptable salt thereof can be about 12.5 mg. The agent to support one or more biogenic amines can be selected from the group consisting of L-DOPA, 5-HTP, EGCG, and N-acetyl-cysteine, or pharmaceutically acceptable salts thereof. The therapeutically effective amount of 5-HTP or a pharmaceutically acceptable salt thereof can be between about 5 mg and about 10,000 mg. The therapeutically effective amount of 5-HTP or a pharmaceutically acceptable salt thereof can be about 100 mg. The therapeutically effective amount of L-DOPA or a pharmaceutically acceptable salt thereof can be between about 10 mg to about 20,000 mg. The therapeutically effective amount of L-DOPA or a pharmaceutically acceptable salt thereof can be about 100 mg. The therapeutically effective amount of EGCG or a pharmaceutically acceptable salt thereof can be between about 10 mg and about 10,000 mg. The therapeutically effective amount of EGCG or a pharmaceutically acceptable salt thereof can be about 150 mg. The decarboxylase inhibitor can be selected from the group consisting of EGCG, carbidopa, benserazide, difluoromethyldopa, and α-methyldopa, or pharmaceutically acceptable salts thereof. The decarboxylase inhibitor can be EGCG. The therapeutically effective amount of EGCG or a pharmaceutically acceptable salt thereof can be between about 10 mg and about 10,000 mg. The therapeutically effective amount of EGCG or a pharmaceutically acceptable salt thereof can be about 150 mg.

In another aspect, this document features a method of reducing cravings associated with initiating a behavior modification program in a human. The method can comprise administering to the human an effective amount of a composition comprising an agent to support acetylcholine function and an agent to support one or more biogenic amines.

In another aspect, this document features a method of reducing cravings associated with initiating a behavior modification program in a human. The method can comprise administering to a human an effective amount of a composition comprising Huperzine A, forskolin, 5-HTP, N-acetyl-cysteine, L-DOPA, and EGCG, or pharmaceutically acceptable salts thereof. The behavior modification program can be a smoking reduction or cessation program. The behavior modification program can be a program to treat obsessive-compulsive disorder. The behavior modification program can be a chemical dependency program. The behavior modification program can be a detoxification program.

In a further aspect, this document features a method of reducing cravings associated with reducing or eliminating the use of a chemical substance in a human. The method can comprise administering to the human an effective amount of a composition comprising an agent to support acetylcholine and an agent to support one or more biogenic amines. The chemical substance can be selected from the group consisting of nicotine, opioids, methamphetamine, cannabis, and alcohol.

In another aspect, this document features a method of reducing cravings associated with reducing or eliminating the use of a chemical substance in a human. The method can comprise administering to said human an effective amount of a composition comprising Huperzine A, forskolin, 5-HTP, N-acetyl-cysteine, L-DOPA, and EGCG, or pharmaceutically acceptable salts thereof. The chemical substance can be selected from the group consisting of nicotine, opioids, methamphetamine, cannabis, and alcohol.

In another aspect, this document features a method of supporting appetite control in a human. The method can comprise administering to the human an effective amount of a composition comprising an agent to support acetylcholine and an agent to support one or more biogenic amines. The human can be reducing or eliminating nicotine intake. The human can be attempting to reduce or eliminate nicotine intake. The human can be reducing or eliminating smoking. The human can be attempting to reduce or eliminate smoking.

In a further aspect, this document features a method of supporting appetite control in a human. The method can comprise administering to the human an effective amount of a composition comprising Huperzine A, forskolin, 5-HTP, N-acetyl-cysteine, L-DOPA, and EGCG, or pharmaceutically acceptable salts thereof. The human can be reducing or eliminating nicotine intake. The human can be attempting to reduce or eliminate nicotine intake. The human can be reducing or eliminating smoking. The human can be attempting to reduce or eliminate smoking

In another aspect, this document features a method of treating appetite disturbance associated with initiating a smoking reduction or cessation program in a human. The method can comprise administering to the human an effective amount of a composition comprising Huperzine A, forskolin, 5-HTP, N-acetyl-cysteine, L-DOPA, and EGCG, or pharmaceutically acceptable salts thereof.

In another aspect, this document features a method of preventing weight gain associated with reducing and/or eliminating nicotine intake in a human. The method can comprise administering to the human an effective amount of a composition comprising an agent to support acetylcholine and an agent to support one or more biogenic amines.

In a further aspect, this document features a method of preventing weight gain associated with reducing and/or eliminating nicotine intake in a human. The method can comprise administering to the human an effective amount of a composition comprising Huperzine A, forskolin, 5-HTP, N-acetyl-cysteine, L-DOPA, and EGCG, or pharmaceutically acceptable salts thereof.

In another aspect, this document features a method of preventing or reducing weight gain associated with smoking reduction or cessation in a human. The method can comprise administering to the human an effective amount of a composition comprising an agent to support acetylcholine and an agent to support one or more biogenic amines.

In another aspect, this document features a method of preventing or reducing weight gain associated with smoking reduction or cessation in a human. The method can comprise administering to the human an effective amount of a composition comprising Huperzine A, forskolin, 5-HTP, N-acetyl-cysteine, L-DOPA, and EGCG or pharmaceutically acceptable salts thereof.

In a further aspect, this document features a method of treating appetite disturbance associated with reducing and/or eliminating nicotine intake in a human. The method can comprise administering to the human an effective amount a composition comprising an agent to support acetylcholine and an agent to support one or more biogenic amines.

In another aspect, this document features a method of treating appetite disturbance associated with reducing and/or eliminating nicotine intake in a human. The method can comprise administering to said human an effective amount of a composition comprising Huperzine A, forskolin, 5-HTP, N-acetyl-cysteine, L-DOPA, and EGCG, or pharmaceutically acceptable salts thereof.

In another aspect, this document features a method of treating appetite disturbance associated with initiating a smoking reduction or cessation program in a human. The method can comprise administering to the human an effective amount of a composition comprising an agent to support acetylcholine function and an agent to support one or more biogenic amines. The composition can support healthy efforts to lose weight. The composition can maintain efficient metabolism in the body. The composition can support a balanced appetite. The composition can facilitate weight loss or weight maintenance. The composition can reduce Body Mass Index (BMI) of the human. The composition can enhance a feeling of satiety in the human. The composition can reduce cravings for a chemical substance. The composition can support efforts to reduce or eliminate addictive behavior. The composition can support efforts to reduce or eliminate impulsive behavior or reduce or eliminate obsessive behavior.

In another aspect, this document features an article of manufacture. The article of manufacturing can comprise a composition and instructions describing a method of using the composition for supporting a balanced appetite before, during, or after a smoking reduction or cessation regimen or a nicotine reduction regimen.

In another aspect, this document features an article of manufacture. The article of manufacture can comprise a composition and instructions describing a method of using the composition for reducing Body Mass Index (BMI) before, during, or after a smoking reduction or cessation regimen or a nicotine reduction regimen.

In another aspect, this document features an article of manufacture. The article of manufacture can comprise a composition and instructions describing a method of using the composition for facilitating weight loss or weight maintenance before, during, or after a smoking reduction or cessation regimen or a nicotine reduction regimen.

In another aspect, this document features an article of manufacture. The article of manufacture can comprise a composition and instructions describing a method of using the composition for controlling food cravings before, during, or after a smoking reduction or cessation regimen or a nicotine reduction regimen.

In a further aspect, this document also features an article of manufacture. The article of manufacture can comprise a composition. The composition can be in an oral dosage form. The article of manufacture can comprise a composition within a pill, a tablet, a capsule, or a syringe dosage form.

In another aspect, this document features use of a composition in the preparation of a therapeutic agent for the prevention or reduction of weight gain in a human. The weight gain can be associated with a smoking reduction or cessation regimen or a nicotine reduction regimen.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the description below. Other features, objects, and advantages of the invention will be apparent from the description and from the claims.

### DETAILED DESCRIPTION

This document provides methods and materials related to reducing cravings and impulses associated with addictive and compulsive behaviors. For example, provided herein are compositions or kits containing a combination of an agent to support acetylcholine and an agent to support biogenic amines, and methods for using a composition or kit as described herein for controlling cravings associated with behavior modification programs, addictive and/or compulsive behaviors (e.g., gambling, sex, and repetitive behaviors), or the reduction or cessation of the use of chemical substances (e.g., nicotine, tobacco, drugs of abuse, including opioids, cocaine, methamphetamine, and alcohol). As described herein, the methods and materials may also be used to prevent or reduce weight gain, control appetite and cravings, reduce the perception of hunger, and promote a healthy weight associated with smoking reduction or cessation regimens and/or nicotine reduction or cessation regimens. For example, compositions comprising an agent to support acetylcholine and an agent to support biogenic amines, and methods for using such compositions to prevent or reduce cravings, prevent or reduce weight gain, and control appetite are provided. While not being bound by any theory, compositions provided herein can induce satiety, aid in weight loss, aid in glycemic control, and decrease cravings associated with behavior modification programs such as smoking reduction or cessation regimens and/or nicotine reduction or cessation regimens. Compositions provided herein can also decrease cravings associated with reduction or cessation of the use of chemical substances, behavior modification protocols, and addictive and/or compulsive behaviors.

As used herein, the term “treat” means to decrease, suppress, attenuate, diminish, arrest, or stabilize the development or progression of a disease or disorder delineated herein (e.g., weight gain, obesity, cravings associated with a behavior modification program), lessen the severity of the disease or disorder, or improve the symptoms associated with the disease or disorder.

The term “prevent” refers to reducing the likelihood of developing a disease or disorder delineated herein (e.g., preventing weight gain associated with reducing and/or eliminating smoking or nicotine intake).

A salt of a compound can be formed between an acid and a basic group of the compound, such as an amino functional group, or a base and an acidic group of the compound, such as a carboxyl functional group. A compound used in a composition or kit herein can be a salt, e.g., a pharmaceutically acceptable salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any suitable salt that, upon administration to a recipient, is capable of providing, either directly or indirectly, a compound as described herein. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne 1,4 dioate, hexyne 1,6 dioate, benzoate, chlorobenzoate, methylbenzoate, dinitrobenzoate, hydroxybenzoate, methoxybenzoate, phthalate, terephathalate, sulfonate, xylene sulfonate, phenylacetate, phenylpropionate, phenylbutyrate, citrate, lactate, β hydroxybutyrate, glycolate, maleate, tartrate, methanesulfonate, propanesulfonate, naphthalene 1 sulfonate, naphthalene 2 sulfonate, mandelate and other salts.

### Compositions

Compositions described herein include an effective amount of an agent to support acetylcholine and an agent to support one or more biogenic amines. Such compounds can be used to support acetylcholine by maintaining or increasing endogenous acetylcholine accumulation, biosynthesis, and activity. In some cases, two or more of the agents described above can be admixed to result in a composition of a single dosage form. For example, compositions described herein can comprise an admixture of an agent to support acetylcholine and an agent to support one or more biogenic amines in a single dosage form.

In some cases, two or more of the agents described above can be presented in a separate dosage form as a kit. The term “kit” as used herein means that the separate dosage forms are packaged together or otherwise associated with one another or attached to one another such that it is readily apparent that the separate dosage forms are intended to be sold and administered together (within less than 24 hours of one another, consecutively or simultaneously). For example, kits as described herein can include a separate dosage form of an agent to support acetylcholine associated with a separate dosage form of an agent to support biogenic amines.

An appropriate agent to support acetylcholine for use in compositions described herein can be an acetylcholine receptor activating compound, an acetylcholine precursor, or an agent that functions to inhibit enzymatic breakdown of acetylcholine (e.g., an acetylcholinesterase inhibitor). Supporting acetylcholine can include maintaining or increasing acetylcholine levels, or promoting or increasing acetylcholine function. For example, an agent to support acetylcholine can include alpha glycerylphosphorylcholine, citicholine, choline, acetylcarnitine, centrophenoxine, dimethylaminoethanol, physostigmine, neostigmine, pyridostigmine, ambenonium, demarcarium, rivastigmine, phenanthrene derivatives, galantamine, donepezil, tacrine, edrophonium, Huperzine A, donepezil, and diisopropyl phosphorofluoridate. In some embodiments, an anticholinesterase compound can be the acetylcholinesterase (AChE) inhibitor Huperzine A. Huperzine A (HupA) is found in the leaves and purified extract of the plant Huperzia serrata and also can obtained from commercial suppliers (e.g., available from Sigma Aldrich). In other cases, an agent to support acetylcholine function can be an inhibitor of an enzyme related to AChE called butyrylcholinesterase (BChE). It has been observed that BChE can substitute for acetylcholinesterase by hydrolyzing the neurotransmitter acetylcholine. See Duysen et al., Expert Opin. Drug Metab. Toxicol. 5(5):523-8 (2009).

An appropriate agent to support one or more biogenic amines for use in the compositions described herein can be, for example, a neurotransmitter precursor, (e.g., L-DOPA, 5-HTP), an amino acid or an amino acid derivative (e.g., N-acetyl-cysteine, cysteine, N-acetyl-tyrosine, tyrosine, D,L-phenylalanine, L-phenylalanine, L-histidine, L-theanine, L-tryptophan, dipeptide, tripeptide, or oligopeptide amino acid, or pharmaceutically acceptable salts thereof). The dopamine precursor L-DOPA is found in fava, the extract of the plant Vicia faba, and is commercially available as a purified compound (e.g., from Sigma Aldrich). L-DOPA also is found in plants of the genus Mucuna (e.g. Mucuna pruriens, Mucuna cochinchinensis). Fava has been shown to increase serum levels of DOPAC, norepinephrine, and levodopa, which is converted into dopamine in situ.

In some cases, an agent to support one or more biogenic amines can be epigallocatechin gallate (EGCG). EGCG has been demonstrated to reduce the perception of appetite and decrease food intake. See Kao et al., Am. J. Clin. Nutr. 72:1232-1234 (2000). EGCG also has been described to increase lipid digestion, exhibit strong anti-inflammatory activity, and increase thermogenetic activity. See, e.g., Dulloo et al., Am. J. Chin. Nutr. 70:1040-1045 (1999). By increasing serum levels of norepinephrine and decreasing the activity of enzymes responsible for degrading catecholamines such as dopamine, epinephrine, and norepinephrine, EGCG potentiates the effect of catecholamine precursors. See Bertoldi et al., Biochem. Biophys. Res. Commun. 284(1):90-93 (2001).

In some cases, agents to support one or more biogenic amines can include the serotonin precursor 5-hydroxytryptophan (5-HTP) or N-acetyl-cysteine (NAC). NAC is a sulfur-containing amino acid which has been found to normalize glutamate signaling and reduce some addictive behaviors. Other amino acids or amino acid derivatives appropriate for compositions described herein can include, for example, cysteine, N-acetyl-tyrosine, L-tyrosine, D,L-phenylalanine, taurine, glycine, L-phenylalanine, L-methionine, selenomethionine, L-histidine, L-glutamine, L-theanine, 4-amino-3-phenylbutyric acid, L-glutamate-γ-ethyl ester, L-glutamate-γ-hydrazide, poly-L-glutamate, poly-L-glycine, 4-amino-3-(4-chlorophenyl)-butanoic acid (baclofen), L-serine, β-alanine, L-tryptophan, L-tryptamine, 5-hydroxytryptamine (5-HT), 5-hydroxytryptophol (5-HTOL), and N-acetyl-5-hydroxytryptamine (N-Ac-5-HT), or an O-phospho amino acid derivative, methyl ester amino acid derivative, or pharmaceutically acceptable salts thereof.

In some cases, an agent to support one or more biogenic amines can be a decarboxylase inhibitor such as EGCG, carbidopa, benserazide, difluoromethyldopa, and α-methyldopa. Further improvement in biogenic amines can be achieved by inhibiting phosphodiesterase and catechol-O-methyl-transferase (COMT), enzymes which support second messenger signaling and catecholamines, respectively. EGCG is believed to exert thermogenetic and antiobesity effects via the inhibition of phosphodiesterase and catechol O-methyl-transferase (COMT). Dulloo et al., Am. J. Clin. Nutr. 70:1040-45 (1999).

In other cases, the agent to support one or more biogenic amines can be a medication. In some cases the agent to support one or more biogenic amine can be monoamine reuptake inhibitor (MRIs), selective serotonin reuptake inhibitors (SSRIs), serotonin-norepinephrine reuptake inhibitors (SNRIs), norepinephrine reuptake inhibitors (NRIs), norepinephrine-dopamine reuptake inhibitors (NDRIs), monoamine releasing agents (MRAs), norepinephrine-dopamine releasing agents (NDRAs), tricyclic antidepressant (TCAs), tetracyclic antidepressants (TeCAs), monoamine oxidase inhibitors (MAOIs), 5-HT1A Receptor Agonists, 5-HT2 Receptor Antagonists, Selective Serotonin Reuptake Enhancers (SSREs), sigma receptor agonists, and anticonvulsant.

Compositions provided herein can comprise, or consist essentially of, a therapeutically effective amount of two or more (2, 3, 4, 5, 6, 7, 8, or more) of the agents described herein, e.g., two or more of an agent to support acetylcholine; or two or more of an agent to support one or more biogenic amines, or a pharmaceutically acceptable salt of such compounds; and in some embodiments, an acceptable carrier.

In some cases, compositions provided herein additionally can comprise a therapeutically effective amount of one or more components such as, for example, chromium polynicotinate (niacin-bound chromium), vitamin B5 (pantothenic acid), L-glutamine, vitamin B9 (folic acid), vitamin B6, and vitamin C (L-ascorbic acid), or derivatives thereof.

In some embodiments, compositions described herein can additionally include a therapeutically effective amount of an agent to support second messenger signal transduction. An appropriate agent to support second messenger signal transduction can be, for example, forskolin. Forskolin is a specific and reversible activator of adenylate cyclase. Forskolin is extracted from the plant Coleus forskohlii and is available as a purified compound from commercial suppliers (e.g., available from Sigma Aldrich). Forskolin directly activates adenylate cyclase and increases intracellular cyclic adenosine monophosphate (cAMP) levels. In some cases, EGCG can be used to support second messenger signal transduction. See Chou et al., Synapse 61(11):889-902 (2007). In other cases, an appropriate agent to support second messenger signal transduction can be sclareolide, which is a diterpene cAMP activator. Other appropriate agents to support second messenger signal transduction can include adenyl cyclase activators such as benzyloxybenzaldehyde analogs. See Chang et al., Bioorganic & Medicinal Chem. Letters 11(15):1971-74 (2001). In some cases, agents to support second messenger signal transduction can be chemical chaperones such as tauroursodeoxycholic acid (TUDCA) or tetrabutylammonium (TBA). Support of second messenger signaling mechanism can be achieved with agents that target further aspects of the second messenger systems (e.g., cyclases, phospholipases, kinases). In some cases, a composition can include two or more (2, 3, 4, 5, 6, 7, 8, or more) agents to support second messenger signal transduction as described above.

A composition can be formulated for pharmaceutical use (“a pharmaceutical composition”), wherein the carrier is a pharmaceutically acceptable carrier. The carrier(s) are “acceptable” in the sense of being compatible with the other ingredients of the formulation and, in the case of a pharmaceutically acceptable carrier, not deleterious to the recipient thereof in an amount used in the composition. Pharmaceutically acceptable carriers, adjuvants and vehicles that may be used in the compositions described herein can include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

Use of any of the compositions provided herein, or their pharmaceutically acceptable salts or derivatives, to control cravings associated with reduction or cessation of the use of chemical substances (e.g., nicotine, drugs of abuse including opioids, cocaine, methamphetamine, alcohol), to reduce cravings associated with addictive and/or compulsive behaviors (e.g., gambling, sex, and repetitive behaviors) is provided.

Use of any of the compositions provided herein, or their pharmaceutically acceptable salts or derivatives, to reduce appetite and to prevent or reduce weight gain, obesity, or other weight-related disorders associated with reduction or cessation of smoking and/or reduction or cessation of nicotine intake is also provided.

Use of any of the compositions provided herein, or their pharmaceutically acceptable salts or derivatives, in the preparation of a medicament or other therapeutic agent for the treatment or amelioration of cravings while participating in a smoking cessation program, nicotine reduction program, or behavior modification program is also provided.

### Formulation of Pharmaceutical Compositions

The pharmaceutical compositions provided herein are useful in the treatment, prevention, or amelioration of cravings associated with reduction or cessation of addictive and/or compulsive behaviors (e.g., gambling, sex, repetitive behaviors), with reduction or cessation of the use of chemical substances, and with behavioral modification programs (e.g., smoking cessation or reduction protocol, nicotine reduction program). Pharmaceutical compositions are also useful in the treatment, prevention, or amelioration of one or more of the weight-related symptoms associated with reducing or eliminating smoking and/or nicotine intake. Pharmaceutical carriers suitable for administration of the compounds provided herein include any such carriers known to those skilled in the art to be suitable for the particular mode of administration. In some cases, the compounds may be formulated as the sole pharmaceutically active ingredients in the composition, or may be combined with other active ingredients.

Pharmaceutical carriers suitable for administration of the compounds provided herein can include carriers known to those skilled in the art to be suitable for the particular mode of administration. In addition, the compounds can be formulated as the sole pharmaceutically active ingredient in the composition or may be combined with other active ingredients. The active compounds can be included in the pharmaceutically acceptable carrier in an amount sufficient to exert a therapeutically useful effect in the absence of undesirable side effects on the patient treated. In some cases, the therapeutically effective concentration can be determined empirically by testing the compounds in in vitro and in vivo systems, and then extrapolated therefrom for dosages for humans.

The compositions are, in one embodiment, formulated into suitable pharmaceutical preparations such as tablets, dispersible tablets, pills, capsules, powders, sustained release formulations, solutions, suspensions, or elixirs for oral administration or in sterile solutions or suspensions for parenteral administration, as well as transdermal patch preparation and dry powder inhalers. In one embodiment, the compounds described above are formulated into pharmaceutical compositions using techniques and procedures well known in the art (see, e.g., Ansel Introduction to Pharmaceutical Dosage Forms, Fourth Edition 1985, 126).

In some cases, the preferable route of administration can be oral. Compositions and kits provided herein suitable for oral administration can be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. The tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating. Examples of binders include microcrystalline cellulose, gum tragacanth, glucose solution, acacia mucilage, gelatin solution, molasses, polyinylpyrrolidine, povidone, crospovidones, sucrose, and starch paste. Lubricants include talc, starch, magnesium or calcium stearate, lycopodium and stearic acid. Diluents include, for example, lactose, sucrose, starch, kaolin, salt, mannitol, and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

The compositions could be provided in a composition in a form that protects it from the acidic environment of the stomach. For example, the composition can be formulated in an enteric coating that maintains its integrity in the stomach and releases the active compound in the intestine. The composition also can be formulated in combination with an antacid or other such ingredient.

When the dosage unit form is a capsule, it can contain, in addition to material of the above type, a liquid carrier such as fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors. Soft gelatin capsules can be useful for containing suspensions, which may beneficially increase the rate of compound absorption. In some cases, capsules for oral use can include vegetable cellulose, microcrystalline, or carob, which is void of any animal derivatives. The compositions and kits described herein can be hypoallergenic.

The active materials can also be mixed or complexed with other active materials which do not impair the desired action, or with materials that supplement the desired action. The active ingredient is a compound or pharmaceutically acceptable derivative thereof as described herein. Higher concentrations, up to about 98% by weight of the active ingredient, may be included.

If required, the solubility and bioavailability of the compounds provided herein can be enhanced by methods well-known in the art. One such method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, (2006). Other methods of enhancing bioavailability can include use of an amorphous form of a compound of this invention optionally formulated with a poloxamer, such as LUTROL™ and PLURONIC™ (BASF Corporation), or block copolymers of ethylene oxide and propylene oxide. See U.S. Pat. No. 7,014,866; and United States Patent Pub. US2006/0094744 and US2006/0079502. In some cases, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

Compositions described herein can include those suitable for oral, rectal, nasal, topical (including buccal and sublingual), vaginal, or parenteral (including subcutaneous, intramuscular, intravenous and intradermal) administration. In some cases, the composition or kit as described herein is administered transdermally (e.g., using a transdermal patch or iontophoretic techniques). Actual methods of preparing other such dosage forms (e.g., parenteral or transdermal dosage forms, sustained release capsules, liposomes) are known, or will be apparent, to those skilled in this art. See, for example, Remington's Pharmaceutical Sciences, Mack Publishing Company, Philadelphia, Pa. (17th ed. 1985).

When aqueous suspensions are administered orally, the active ingredient(s) may be combined with emulsifying and suspending agents. In some embodiments, a composition or kit as described herein can be mixed with soft food (e.g., yogurt, pudding, apple sauce, oatmeal, or baby food) for oral administration. If desired, certain sweetening and/or flavoring and/or coloring agents may be added (e.g., fructose and lemon, rosemary, or peppermint oil). Compositions suitable for oral administration include lozenges comprising the ingredients in a flavored basis, such as sucrose and acacia or tragacanth; and pastilles comprising the active ingredient in an inert basis such as vegetable cellulose, gelatin and glycerin, or sucrose and acacia. Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Compositions for injection can be in the form, for example, of a sterile injectable aqueous or oleaginous solution or suspension. This suspension can be formulated according to techniques known in the art using suitable dispersing or wetting agents (such as, for example, TWEEN® 80) and suspending agents. Such sterile injectable preparations can also be a sterile injectable solution or suspension in a non-toxic parenterally-acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils can be conventionally employed as a solvent or suspending medium. For this purpose, any bland fixed oil can be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. Such oil solutions or suspensions can also contain a long-chain alcohol diluent or dispersant.

The compositions described herein can be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax, and polyethylene glycols.

The compositions described herein can be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, e.g., Rabinowitz and Zaffaroni, U.S. Pat. No. 6,803,031.

The compositions described herein can be administered topically. For topical application topically to the skin, the pharmaceutical composition should be formulated with a suitable ointment containing the active components suspended or dissolved in a carrier. Carriers for topical administration of the compositions and kits described herein can include, but are not limited to, mineral oil, liquid petroleum, white petroleum, propylene glycol, polyoxyethylene polyoxypropylene compound, emulsifying wax, and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol, and water. The pharmaceutical compositions provided herein may also be topically applied to the lower intestinal tract by rectal suppository formulation or in a suitable enema formulation. Topically-transdermal patches and iontophoretic administration are also included in this invention.

In some cases, a composition as described herein further comprises a second therapeutic agent. The second therapeutic agent may be selected from any compound or therapeutic agent known to have or that demonstrates advantageous properties when administered with a compound having the same mechanism of action of an agent to support acetylcholine, an agent to support biogenic amines, or an agent to support second messenger signal transduction, e.g., a second anticholinesterase compound as described above; an anti-inflammatory agent (e.g., non-steroidal anti-inflammatory drugs, broad spectrum chemokine inhibitors, and glucocorticoids); an anti-oxidant agent (e.g., resveratrol, flavonoids, carotenoids, glutathione, co-enzyme Q10, idebenone, and ubiquinone); a cholinomimetic agent (e.g., bethanechol, pilocarpine, and cevimeline), a botanical or botanical extract (e.g., Mucuna pruriens, Griffonia simplicifolia, Boswellia serrata, Rhodiola rosea, and Stevia rebaudiana); a nutritional or dietary supplement (e.g., inositol, creatinine, Krill oil, fish protein hydrolysase, lecithin or phosphatidylserine enriched soy lecithin, alpha-lipoic acid, docosahexaenoic acid, eicosapentaenoic acid, and alpha-linoleic acid); a mineral (e.g., chromium, calcium, magnesium, zinc, selenium, manganese, molybdenum, and iodine); or a vitamin (e.g., vitamin A (beta carotene or retinal acetate), vitamin C (ascorbic acid), vitamin D (cholecalciferol), vitamin E (d-alpha tocopherol succinate), thiamine, riboflavin, niacin (niacinamide), vitamin B6 (pyroxidine HCl or pyrodoxal 5′ phosphate), vitamin B12, biotin, folic acid (folacin), and pantothentic acid (calcium pantothene)).

In some cases, compositions described herein can be provided in a dosage form and provided with one or more of any of the above-described second therapeutic agents in a separate dosage form, wherein the composition and second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

As described herein, the compositions are administered in an effective amount. As used herein, the term “effective amount” refers to an amount which, when administered in a proper dosing regimen, is sufficient to reduce or ameliorate the severity, duration, or progression of the disorder being treated, prevent the advancement of the disorder being treated, cause the regression of the disorder being treated, enhance or improve the prophylactic or therapeutic effect(s) of another therapy, or to promote a healthy weight.

The interrelationship of dosages for animals and humans (based on milligrams per meter squared of body surface) is described in Freireich et al., Cancer Chemother. Rep. 50:219 (1966). Body surface area can be approximately determined from height and weight of the subject. See, e.g., Scientific Tables, Geigy Pharmaceuticals, Ardsley, N.Y., 1970, 537.

An effective amount of an agent to support acetylcholine can range between about 20 micrograms (μg) and about 5000 μg (e.g., between about 20 μg and about 500 μg; about 50 μg and about 500 μg; about 100 μg and about 750 μg; about 100 μg and about 1000 μg; about 250 μg and about 1000 μg; about 1000 μg and about 2500 μg; about 1000 μg and about 5000 μg). In some cases, an effective amount of an agent to support acetylcholine can be about 240 μg. In some cases, an effective amount of an agent to support acetylcholine can be about 200 μg.

In some cases, the agent to support acetylcholine is Huperzine A, or a pharmaceutically acceptable salt thereof. Effective daily dosages of Huperzine A, or a pharmaceutically acceptable salt thereof, can range between about 20 μg and about 5000 μg. In other cases, the agent to support acetylcholine can be alpha glycerylphosphorylcholine, physostigmine, neostigmine, pyridostigmine, ambenonium, demarcarium, rivastigmine, phenanthrene derivatives, galantamine, donepezil, tacrine, edrophonium, donepezil, or diisopropyl phosphorofluoridate, or pharmaceutically acceptable salts thereof.

An effective amount of an agent to support one or more biogenic amines can range between about 5 mg and about 100,000 mg (e.g., between about 5 mg and about 100,000 mg; about 5 mg and about 50,000 mg; about 10 mg and about 50,000 mg; about 20 mg and about 10,000 mg; about 50 mg and about 5,000 mg; about 100 mg and about 2,500 mg; about 100 mg and about 1,000 mg). In some cases, an effective amount of an agent to support one or more biogenic amines can be about 900 mg. In some cases, an effective amount of an agent to support one or more biogenic amines can be about 100 mg. In some cases, an effective amount of an agent to support one or more biogenic amines can be about 150 mg.

In some cases, the agent to support one or more biogenic amines is 5-HTP. Effective daily dosages of 5-HTP can range between about 5 mg and about 10,000 mg (e.g., between about 5 mg and 10,000 mg, 25 mg and about 2000 mg, about 50 mg and about 2000 mg, about 50 mg and about 1500 mg). In some cases, an effective daily dose of 5-HTP can range between about 25 mg to about 2500 mg. In some cases, an effective daily dose of 5-HTP can be about 100 mg. In some cases, the agent to support one or more biogenic amines is N-acetyl-cysteine in an amount between about 100 mg and about 100,000 mg. An effective daily dose of N-acetyl-cysteine can be about 2700 mg. In some cases, the agent to support one or more biogenic amines is L-DOPA in an amount between about 10 mg to about 20,000 mg. An effective daily dose of L-DOPA can be about 300 mg. In some cases, the agent to support one or more biogenic amines is EGCG in an amount between about 10 mg and about 10,000 mg. An effective daily dose of EGCG can be about 450 mg of EGCG.

In other cases, the agent to support one or more biogenic amines can be a medication. In some cases the agent to support one or more biogenic amine can be monoamine reuptake inhibitor (MRIs), selective serotonin reuptake inhibitors (SSRIs), serotonin-norepinephrine reuptake inhibitors (SNRIs), norepinephrine reuptake inhibitors (NRIs), norepinephrine-dopamine reuptake inhibitors (NDRIs), monoamine releasing agents (MRAs), norepinephrine-dopamine releasing agents (NDRAs), tricyclic antidepressant (TCAs), tetracyclic antidepressants (TeCAs), monoamine oxidase inhibitors (MAOIs), 5-HT1A Receptor Agonists, 5-HT2 Receptor Antagonists, Selective Serotonin Reuptake Enhancers (SSREs), sigma receptor agonists, and anticonvulsant.

In other cases, the agent to support one or more biogenic amines can be N-acetyl-tyrosine, L-tyrosine, D,L-phenylalanine, taurine, glycine, L-phenylalanine, L-methionine, selenomethionine L-histidine, L-glutamine, L-theanine, 4-amino-3-phenylbutyric acid, L-glutamate-γ-ethyl ester, L-glutamate-γ-hydrazide, poly-L-glutamate, poly-L-glycine, 4-amino-3-(4-chlorophenyl)-butanoic acid (baclofen), L-serine, β-alanine, L-tryptophan, L-tryptamine, 5-hydroxytryptamine (5-HT), 5-hydroxytryptophol (5-HTOL), and N-acetyl-5-hydroxytryptamine (N-Ac-5-HT), an O-phospho amino acid derivative, methyl ester amino acid derivative, or pharmaceutically acceptable salts thereof.

An effective amount of an agent to support second messenger signal transduction can range between about 0.5 mg and about 2,000 mg (e.g., between about 0.5 mg and about 2,000 mg; about 0.5 mg and about 1,500 mg; about 1.0 mg and about 1,000 mg; about 5 mg and about 500 mg; about 10 mg and about 500 mg; about 12.5 mg and about 250 mg; about 15 mg and about 100 mg). In some cases, an effective amount of an agent to support second messenger signal transduction can be about 12.5 mg.

In some cases, a composition can additionally include a therapeutically effective amount of an agent to support second messenger signaling. For example, a composition can additionally include forskolin as the agent to support second messenger signal transduction. Effective daily dosages of forskolin can range between about 0.5 mg and about 2,000 mg. In some cases, an effective daily dose of forskolin can range between about 2.5 mg and about 1,000 mg (e.g., about 2.5, 5, 10, 15, 20, 30, 40, 50, 100, 200, 400, 500, 600, 700, 800, 900, or 1,000 mg). In other cases, the agent to support second messenger signal transduction can be sclareolide, tauroursodeoxycholic acid, or tetrabutylammonium, or pharmaceutically acceptable salts thereof.

A composition can be given, for example, one, two, three, four, or more times daily depending on various factors recognized by those skilled in the art. In some cases, one, two, or three separate unit doses of a composition can be given once, twice, or more times per day. For example, a composition comprising 240 micrograms (μg) Huperzine A, 12.5 mg forskolin, 900 mg N-acetyl-cysteine, 100 mg 5-HTP, 100 mg L-DOPA, and 150 mg EGCG can be given three times daily to provide a subject with a total daily amount of 720 μg Huperzine A, 37.5 mg forskolin, 2700 mg N-acetyl-cysteine, 300 mg 5-HTP, 300 mg L-DOPA, and 450 mg EGCG.

Effective amounts will also vary, as recognized by those skilled in the art, depending on the disease or disorder treated, the severity of the disease, the route of administration, the sex, age and general health condition of the subject, excipient usage, the possibility of co-usage with other therapeutic treatments such as use of other agents and the judgment of the treating physician, clinician, or other healthcare provider. For example, guidance for selecting an effective dose can be determined by reference to the pharmacokinetic information for Huperzine A, forskolin, L-DOPA, 5-HTP, N-acetyl-cysteine, and EGCG.

For compositions or kits that comprise a second therapeutic agent, an effective amount of the second therapeutic agent is between about 0.01% to about 100% of the amount normally utilized in a monotherapy regime using just that agent. In some cases, a second therapeutic agent can be chromium polynicotinate (niacin-bound chromium) at an effective amount of about 200 micrograms (μg). In other cases, second therapeutic acids can be vitamin B5 (pantothenic acid) at an effective amount of about 60 mg, vitamin B9 (folic acid) at an effective amount of about 133 μg, vitamin B6 at an effective amount of 6 mg, vitamin C (L-ascorbic acid) at an effective amount of about 60 mg, or L-phenylalanine at an effective amount of about 400 mg, or derivatives thereof. The normal monotherapeutic dosages of second therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

It is expected that some of the second therapeutic agents referenced above will act synergistically with the compounds of this invention. When this occurs, it will allow the effective amount of the second therapeutic agent and/or the compound of this invention to be reduced from that required in a monotherapy. This has the advantage of minimizing toxic side effects of either the second therapeutic agent of a compound of this invention, synergistic improvements in efficacy, improved ease of administration or use and/or reduced overall expense of compound preparation or formulation.

### Methods of Using Compositions

This document also provides methods of preventing weight gain, reducing cravings, controlling appetite, and controlling craving associated with reducing or eliminating smoking (e.g., smoking cessation) and/or reducing or eliminating nicotine intake (e.g., nicotine withdrawal or nicotine cravings) with or without the use of a medicament or other therapeutic regime to assist reducing and/or eliminating smoking Symptoms of nicotine withdrawal can include behavioral, emotional, cognitive, and physiological symptoms which emerge upon cessation or reduction of intake of nicotine. While nicotine withdrawal symptoms can be relatively short lived, nicotine cravings can endure long after nicotine cessation. Nicotine cravings can trigger urges that result in relapse. In some cases, cravings for nicotine can be redirected toward food, thereby promoting weight gain.

The methods comprise administering to a subject (e.g., a human) a composition as described herein. The effect of a composition as described herein on weight, appetite, and food cravings can be monitored by subjective (e.g., opinion or self-report of cravings and food intake) and/or objective indicators (e.g., measurable by a test or diagnostic method). Any appropriate method can be used to evaluate the effects of compositions described herein on food cravings. In some cases, the measures used for assessing cravings in clinical settings can include single-item or multiple-item questionnaires. For example, the multidimensional Trait and State Food Cravings Questionnaires (FCQ-T and FCQ-S, respectively) can be used for craving assessment. See Cepeda-Benito et al., Behavior Therapy 31:151-173 (2000). In some cases, the Weight Control Subscale of the Smoking Consequences Questionnaire can be used for food craving assessment. See, e.g., White et al., Addict. Behav. 32(10):2200-2210 (2007); Copeland et al., Psychological Assessment 7:484-494 (1995). The Weight Control Subscale questionnaire assesses weight and nicotine expectancy factors (e.g., weight control, fear of weight gain, weight gained in previous smoking cessation attempt, negative consequences, positive reinforcement, sensory satisfaction, negative reinforcement, negative affect reduction).

Cravings associated with addictive or compulsive behaviors (e.g., an urge or desire to smoke or to use drugs of abuse) can be assessed according to self-reported cravings, which provide a subjective index of a subject's motivational state. In some cases, such cravings can be assessed using one or more multidimensional scales such as the Questionnaire of Smoking Urges (QSU) developed by Tiffany & Drobes (Br. J. Addict. 86(11):1467-76 (1991)) which assesses the subject's desire to smoke and his or her expectancies of both positive and negative reinforcement from smoking and intention to smoke. Other questionnaires or indices useful for assessing cravings for nicotine or smoking can include the Measurement of Drug Craving scale (Sayette et al. 2000), Drug History Questionnaire (DHQ), Desires for Drug Questionnaire (Franken et al., Addict. Behav. 27:675-85 (2002)), Heaviness of Smoking Index (HSI), the Fagerstrom Test for Nicotine Dependence (FTND). In some cases, cravings or impulses associated with addictive and/or compulsive behaviors or behavioral modification protocols can be assessed using the Obsessive-Compulsive Beliefs Questionnaire-87 (OBQ-87).

Baseline craving assessment can be performed prior to administration of a composition provided herein, and additional craving assessments can be performed following administration of the composition. For example, a subject can be asked to report his or her “typical” craving experiences before, during, or after starting a behavioral modification program such as a smoking cessation or reduction program, a nicotine reduction or cessation program (either with or without using a smoking cessation medication (e.g., a transdermal nicotine patch), a program to treat obsessive-compulsive disorder, a chemical dependency program, or a program to reduce or eliminate other addictive and/or compulsive behavior. Baseline craving assessment is useful since individual subjects will differ in the way they use and respond to the questionnaire or other method of assessing the effects of a composition as described herein. Including a baseline score in the analysis can permit normalization of the relevant measures to each subject's standard. The accuracy of craving indices can be limited by the ability and willingness of a subject to accurately report his or her personal experience. Relapse to addictive behavior, frequency of use of nicotine or another addictive substance, the length of time since a subject last smoked or engaged in another addictive behavior, or the length of time since a subject last used nicotine or another addictive substance can also provide meaningful information for evaluating the effects of compositions described herein on weight management and appetite and craving control.

Any appropriate method can be used to objectively evaluate the effects of a composition described herein on craving control, appetite control, impulse control, and weight management. For example, weight can be measured at a time point following administration of compositions described herein and compared to weight measured prior to administration. Similarly, Body Mass Index (BMI) can be calculated based prior to, during, and following administration of compositions described herein and/or prior to, during, and following smoking cessation or using one or more smoking cessation medications. Other methods of determining weight, body fat, and/or changes in weight and body fat over time, can include measuring waist circumference as an indicator of abdominal obesity; performing anthropometry (the skinfold test) which measures skinfold thickness at various body locations; performing Dual Energy X-ray Absorptiometry (DEXA) scan, a low-radiation, full-body X-ray that computes body composition and the percentage of fat in the body by measuring fat mass, lean mass, and bone mass; hydrodensitometry weighing (an underwater weight calculation of body fat); air displacement analysis to provide an estimate of body fat by calculating body density; and bioelectric impedance to provide an estimate of body fat by measuring the body's resistance to a small electrical current. Such methods can be performed by the subject, a clinician, or another health care or fitness professional.

In some cases, the effects of compositions described herein on craving control, weight management, appetite control, or addictive and/or compulsive behavior can be analyzed by assessing a subject prior to, during, or after the subject begins a smoking cessation regime or begins experiencing symptoms associated with nicotine withdrawal or nicotine cravings. In some cases, the effects of compositions described herein on weight management, appetite control, and/or craving control can be analyzed by assessing a subject prior to, during, or after the subject takes or uses, for example, one or more smoking cessation medications, one or more medications to reduce or eliminate use of other chemical substances, or one or more medications to treat obsessive-compulsive disorder.

In some cases, a composition or kit as described herein can be administered to a human to prevent weight gain associated with reducing or eliminating smoking (e.g., smoking cessation) and/or reducing or eliminating nicotine intake (e.g., nicotine withdrawal). Preventing weight gain associated with, for example, smoking cessation can include, for example, promoting loss of fat, increasing lean body mass, increasing metabolism, reducing caloric intake, reducing perception of hunger and/or food cravings, and supporting healthy efforts to lose weight before, during, and/or after smoking reduction or cessation or reduction or withdrawal of nicotine intake. Similarly, preventing weight gain can include promoting loss of fat, increasing lean body mass, increasing metabolism, reducing caloric intake, reducing perception of hunger and/or food cravings, and supporting healthy efforts to lose weight while taking or consuming one or more smoking cessation medications or nicotine replacement compositions.

In some cases, a composition or kit as described herein can be administered to a human to support appetite control before, during, and/or after smoking cessation, nicotine withdrawal or nicotine cravings, or consuming one or more smoking cessation compositions. Supporting appetite control can include reducing perception of appetite cravings, reducing perception of hunger, supporting a balanced appetite, and supporting healthy efforts to lose weight.

In some cases, a composition or kit as described herein can be administered to a human to promote weight management before, during, and/or after smoking cessation, nicotine withdrawal or nicotine cravings, or consuming one or more smoking cessation compositions. Methods of weight management can include inducing satiety, reducing caloric intake, weight loss and improving weight loss, using compositions provided herein.

In some cases, composition or kit described herein can be administered to a human to prevent appetite disturbance associated with smoking cessation, nicotine withdrawal or nicotine cravings, or consuming one or more smoking cessation composition. Preventing appetite disturbance can include reducing perception of appetite cravings, reducing perception of hunger, supporting a balanced appetite, and supporting healthy efforts to lose weight before, during, and/or after smoking cessation, nicotine withdrawal, or consuming one or more smoking cessation compositions.

In some cases, compositions or kits described herein can be used to induce satiety. Inducing satiety can include reducing perception of appetite cravings, reducing perception of hunger, supporting a balanced appetite, and supporting healthy efforts to lose weight before, during, and/or after smoking cessation, nicotine withdrawal or nicotine cravings, or consuming one or more smoking cessation composition.

In other cases, compositions or kits described herein can be used to treat obesity. Treating obesity can include, independently, reducing body weight, preventing obesity in an overweight human, promoting loss of excessive fat (e.g., abdominal fat), reducing the Body Mass Index (BMI) (kg/m²), reducing symptoms associated with obesity (e.g., uncontrolled blood glucose levels, elevated blood pressure, and increased cholesterol levels), preventing progression of metabolic syndrome, and reducing levels of C-reactive protein in blood before, during, and/or after smoking cessation, nicotine withdrawal or nicotine cravings, or consuming one or more smoking cessation compositions.

Compositions or kits described herein can be used to manage weight. Examples of managing weight can include, independently, supporting a healthy body weight, supporting healthy efforts to lose weight with balanced lifestyle, maintaining a healthy body image, maintaining efficient metabolism in the body, supporting the digestive system, promoting the natural breakdown of fats, supporting a balanced appetite, supporting healthy energy levels, promoting nutrient absorption, acting in a supporting capacity to balance mood, reducing or preventing food cravings and comfort eating, facilitating weight loss or weight maintenance, enhancing a feeling of satiety, reducing BMI, and interfering with unhealthy adipose cell signaling.

The methods provided herein also include those wherein the subject is identified as in need of a particular stated treatment. Identifying a subject in need of such treatment can be in the judgment of a subject or a health care professional and can be subjective (e.g., opinion or self-reporting) or objective (e.g., measurable by a test or diagnostic method).

In some cases, any of the above methods of treatment comprises the further step of co-administering to the subject one or more additional second therapeutic agents. The choice of second therapeutic agent may be made from any second therapeutic agent known to be useful for co-administration with an agent to support acetylcholine, an agent to support biogenic amines, and an agent to support second messenger signal transduction. The choice of second therapeutic agent is also dependent upon the particular disease or condition to be treated. Examples of second therapeutic agents that may be employed in the methods described above for use in combination compositions comprising a compound of this invention and a second therapeutic agent can include, without limitation, an antidepressant, stimulant, pain reliever, vitamin, or other nutritional supplement. In some cases, a second therapeutic agent can include one or more additional agents to support acetylcholine, to support biogenic amines, or to support second messenger signal transduction.

The combination therapies of described herein can include co-administering to a subject in need thereof a composition as described herein and an additional therapeutic agent as described above. The term “co-administered” as used herein means that the additional second therapeutic agent may be administered together with a compound or compositions provided herein as part of a single dosage form (such as a composition provided herein comprising a compound described herein and an second therapeutic agent as described above) or as separate, multiple dosage forms. Alternatively, the additional agent may be administered prior to, consecutively with, or following the administration of a compound or composition described herein. In such combination therapy treatment, both the composition or kit as described herein and the second therapeutic agent(s) are administered by conventional methods. The administration of a composition or kit of this invention, comprising both a composition or kit as described herein and a second therapeutic agent, to a subject does not preclude the separate administration of that same therapeutic agent, any other second therapeutic agent or any compound described herein to the subject at another time during a course of treatment.

Effective amounts of these second therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the optimal effective-amount range of a second therapeutic agent.

In some cases, where a second therapeutic agent is administered to a subject, the effective amount of a composition provided herein is less than its effective amount would be where the second therapeutic agent is not administered. In other cases, the effective amount of the second therapeutic agent is less than its effective amount would be where the composition of provided herein is not administered. In this way, undesired side effects associated with high doses of either agent may be minimized. Other potential advantages (including without limitation improved dosing regimens and/or reduced drug cost) will be apparent to those of skill in the art.

In yet another aspect, this document provides the use of a composition as described herein together with one or more of the above-described second therapeutic agents in the manufacture of a medicament, either as a single composition or as separate dosage forms, for treatment or prevention in a subject of a disease, disorder or symptom set forth above. Another aspect of the invention is a composition as described herein for use in the treatment or prevention in a subject of a disease, disorder or symptom thereof delineated herein.

### Articles of Manufacture

The present disclosure also provides articles of manufacture for use with the compositions described herein. These articles of manufacture comprise (a) a composition or kit comprising an agent to support acetylcholine and an agent to support biogenic amines provided as an admixture or as separate components in association as described herein, wherein the composition or kit is in a container; and (b) instructions describing a method of using the composition for weight management, controlling cravings, controlling appetite, controlling impulsive behavior, controlling addictive behavior, and other indications as described above.

An article of manufacture described herein can be used to reduce or eliminate addictive behavior. For example, an article of manufacture can include instructions describing a method of using a composition or kit as described herein to reduce or eliminate addictive behavior. In some cases, an article of manufacture can include information regarding the potential benefits associated with administration of a composition or kit as described herein to treat addictive behavior, such as supporting efforts to reduce or eliminate addictive behavior, promoting a positive mood and positive body image, controlling cravings, controlling impulsive behavior, and reducing or preventing cravings for chemical substances.

In another embodiment, articles of manufacture for use to control cravings are provided. Controlling cravings can include reducing or preventing food cravings or comfort eating, reducing or preventing cravings for chemical substances (e.g., nicotine, alcohol, narcotics, opioids, methamphetamine), reducing or preventing the urge to seek such chemical substances, and supporting efforts to reduce or eliminate addictive or compulsive behaviors.

In another embodiment, articles of manufacture for use to support appetite control are provided. Supporting appetite control can include enhancing the feeling of satiety, supporting a healthy balanced appetite, supporting healthy energy levels and nutrient absorption, and reducing or preventing food cravings or comfort eating. In some cases, an article of manufacture as described herein can be useful in supporting appetite control in a subject participating in a smoking cessation program or experiencing symptoms associated with nicotine withdrawal or nicotine cravings.

An article of manufacture described herein can be used to manage body weight. For example, an article of manufacture can include instructions describing a method of using a composition or kit as described herein to manage weight. In some cases, an article of manufacture as described herein can be useful in managing body weight in a subject participating in a smoking cessation program or experiencing symptoms associated with nicotine withdrawal or nicotine cravings. In some cases, an article of manufacture can include information regarding the potential benefits associated with administration of a composition or kit as described above (e.g., supporting a healthy body weight, supporting healthy efforts to lose weight along with a balanced lifestyle, maintaining healthy weight goals, maintaining efficient metabolism in the body, promoting the natural breakdown of fats, supporting a healthy balanced appetite, supporting healthy energy levels and nutrient absorption, reducing or preventing food cravings and comfort eating, supporting routine weight management and a healthy metabolism, facilitating weight loss or weight maintenance, maintaining efficient metabolism, promoting a positive mood, positive body image, and increased energy, enhancing the feeling of satiety, and reducing Body Mass Index (BMI)).

According to another embodiment, an article of manufacture can include instructions describing a method of using a composition or kit as described herein to treat obesity. In some cases, an article of manufacture as described herein can be useful in treating obesity in a subject participating in a smoking cessation program or experiencing symptoms associated with nicotine withdrawal or nicotine cravings. In some cases, an article of manufacture can include information regarding the potential benefits associated with administration of a composition or kit as described herein to treat obesity, such as reducing body weight, preventing obesity in an overweight human, promoting loss of excessive fat (e.g., abdominal fat), reducing BMI, reducing symptoms associated with obesity (e.g., uncontrolled blood glucose levels, elevated blood pressure, and increased cholesterol levels), preventing progression of metabolic syndrome, and reducing levels of C-reactive protein in blood.

The container may be any vessel or other sealed or sealable apparatus that can hold said pharmaceutical composition. Examples include bottles, ampules, divided or multi-chambered holders bottles, wherein each division or chamber comprises a single dose of said composition, a divided foil packet wherein each division comprises a single dose of said composition, or a dispenser that dispenses single doses of said composition. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example, a conventional cardboard box would not generally be used to hold a liquid suspension. In some cases, more than one container can be used together in a single package to market a single dosage form. For example, tablets may be contained in a bottle, which is in turn contained within a box. In on embodiment, the container is a blister pack.

The articles of manufacture described herein can also comprise a device to administer or to measure out a unit dose of the pharmaceutical composition. Such a device can include an inhaler if the composition is inhalable; a syringe and needle if the composition is injectable; a syringe, spoon, pump, or a vessel with or without volume markings if the composition is an oral liquid composition; or any other measuring or delivery device appropriate to the dosage formulation of the composition present in the articles of manufacture.

In some cases, the articles of manufacture described herein can comprise a separate vessel of container a pharmaceutical composition comprising a second therapeutic agent, such as one of those listed above for use for co-administration with a compound of provided herein.

### EXAMPLES

Table 1 provides therapeutically effective amounts of compounds which can be used to prepare compositions for the indications provided herein. In a preferred embodiment, a composition for the indications provided herein includes the first 6 compounds.

TABLE 1
Formulation

| Compound | Effective Amount | Units |
|---|---|---|
| Huperzine A | 240 | μg |
| L-DOPA | 100 | mg |
| N-acetyl-cysteine | 900 | mg |
| Forskolin | 12.5 | mg |
| 5-HTP | 100 | mg |
| EGCG | 150 | mg |
| L-Phenylalanine | 400 | mg |
| Vitamin B6 (3:1) | 6 | mg |
| 75% pyridoxine HCl | 4.5 | mg |
| 25% pyridoxal 5′-phosphate | 1.5 | mg |
| Vitamin B12 | 133 | μg |
| Pantothenic Acid | 60 | mg |
| Folic Acid | 133 | μg |
| Vitamin C | 60 | mg |
| Chromium Polynicotinate | 200 | μg |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A composition comprising a therapeutically effective amount of an agent to support acetylcholine and an agent to support one or more biogenic amines.

2. A composition comprising a therapeutically effective amount of an agent to support acetylcholine, an agent to support one or more biogenic amines, and an agent to support second messenger signal transduction.

3. The composition of claim 1, further comprising an agent to support second messenger signal transduction.

4. The composition of claim 1, further comprising a therapeutically effective amount of a decarboxylase inhibitor.

5. The composition of claim 1, further comprising one or more of chromium polynicotinate, L-glutamine, folic acid, pantothenic acid, vitamin C, vitamin B6, or L-phenylalanine, or pharmaceutically acceptable salts thereof, or any combination thereof.

6. A composition comprising a therapeutically effective amount of Huperazine A in an amount between about 160 μg and about 240 μg and fluoxetine in an amount between about 5 mg and about 150 mg.

7. The composition of claim 1, further comprising forskolin in an amount between about 0.5 mg and about 15 mg.

8. The composition of claim 1, wherein the agent to support acetylcholine is selected from the group consisting of Huperzine A, alpha glycerylphosphorylcholine, physostigmine, neostigmine, pyridostigmine, ambenonium, demarcarium, rivastigmine, phenanthrene derivatives, galantamine, donepezil, tacrine, edrophonium, donepezil, and diisopropyl phosphorofluoridate, or pharmaceutically acceptable salts thereof.

9. The composition of claim 1, wherein the agent to support one or more biogenic amines is selected from the group consisting of a neurotransmitter precursor, an amino acid or an amino acid derivative, and EGCG, or pharmaceutically acceptable salts thereof.

10. The composition of claim 3, wherein the agent to support second messenger signal transduction is selected from the group consisting of forskolin, sclareolide, a benzyloxybenzaldehyde analog, tauroursodeoxycholic acid, and tetrabutylammonium, or pharmaceutically acceptable salts thereof.

11. The composition of claim 1, wherein the agent to support one or more biogenic amines is selected from the group consisting of 5-hydroxytryptophan (5-HTP), EGCG, L-DOPA, N-acetyl-cysteine, cysteine, N-acetyl-tyrosine, tyrosine, D,L-phenylalanine, L-phenylalanine, L-histidine, L-theanine, L-tryptophan, or pharmaceutically acceptable salts thereof.

12. The composition of claim 1, wherein the composition comprises Huperzine A, forskolin, 5-HTP, N-acetyl-cysteine, L-DOPA, and EGCG, or pharmaceutically acceptable salts thereof.

13. The composition of claim 1, wherein the composition comprises Huperzine A or a pharmaceutically acceptable salt thereof in a range of about 20 μg and about 5000 μg, 5-HTP or a pharmaceutically acceptable salt thereof in a range of about 5 mg and about 10,000 mg, N-acetyl-cysteine or a pharmaceutically acceptable salt thereof in a range of about 100 mg and about 100,000 mg, L-DOPA or a pharmaceutically acceptable salt thereof in a range of about 10 mg to about 20,000 mg, and EGCG or a pharmaceutically acceptable salt thereof in a range of about 10 mg and about 10,000 mg.

14. The composition of claim 1, wherein the agent to support acetylcholine comprises Huperzine A, or a pharmaceutically acceptable salt thereof.

15. The composition of claim 14, wherein the therapeutically effective amount of Huperzine A or a pharmaceutically acceptable salt thereof is between about 20 μg and about 5000 μg.

16. The composition of claim 14, wherein the therapeutically effective amount of Huperzine A or a pharmaceutically acceptable salt thereof is between about 160 μg and about 240 μg.

17. The composition of claim 14, wherein the therapeutically effective amount of Huperzine A or a pharmaceutically acceptable salt thereof is about 240 μg.

18. The composition of claim 3, wherein the agent to support second messenger signal transduction comprises forskolin, or a pharmaceutically acceptable salt thereof.

19. The composition of claim 18, wherein the therapeutically effective amount of forskolin or a pharmaceutically acceptable salt thereof is between about 0.5 mg and about 2,000 mg.

20. The composition of claim 18, wherein the therapeutically effective amount of forskolin or a pharmaceutically acceptable salt thereof is about 12.5 mg.

21. The composition of claim 1, wherein the agent to support one or more biogenic amines is selected from the group consisting of L-DOPA, 5-HTP, EGCG, and N-acetyl-cysteine, or pharmaceutically acceptable salts thereof.

22. The composition of claim 21, wherein the therapeutically effective amount of 5-HTP or a pharmaceutically acceptable salt thereof is between about 5 mg and about 10,000 mg.

23. The composition of claim 21, wherein the therapeutically effective amount of 5-HTP or a pharmaceutically acceptable salt thereof is about 100 mg.

24. The composition of 21, wherein the therapeutically effective amount of L-DOPA or a pharmaceutically acceptable salt thereof is between about 10 mg to about 20,000 mg.

25. The composition of claim 21, wherein the therapeutically effective amount of L-DOPA or a pharmaceutically acceptable salt thereof is about 100 mg.

26. The composition of claim 21, wherein the therapeutically effective amount of EGCG or a pharmaceutically acceptable salt thereof is between about 10 mg and about 10,000 mg.

27. The composition of claim 21, wherein the therapeutically effective amount of EGCG or a pharmaceutically acceptable salt thereof is about 150 mg.

28. The composition of claim 4, wherein the decarboxylase inhibitor is selected from the group consisting of EGCG, carbidopa, benserazide, difluoromethyldopa, and α-methyldopa, or pharmaceutically acceptable salts thereof.

29. The composition of claim 4, wherein the dopa carboxylase inhibitor is EGCG.

30. The composition of claim 29, wherein the therapeutically effective amount of EGCG or a pharmaceutically acceptable salt thereof is between about 10 mg and about 10,000 mg.

31. The composition of claim 29, wherein the therapeutically effective amount of EGCG or a pharmaceutically acceptable salt thereof is about 150 mg.

32. A method of reducing cravings associated with initiating a behavior modification program in a human, the method comprising administering to the human an effective amount of a composition comprising an agent to support acetylcholine function and an agent to support one or more biogenic amines.

33. A method of reducing cravings associated with initiating a behavior modification program in a human, the method comprising administering to the human an effective amount of a composition comprising Huperzine A, forskolin, 5-HTP, N-acetyl-cysteine, L-DOPA, and EGCG, or pharmaceutically acceptable salts thereof.

34. The method of claim 32, wherein the behavior modification program is a smoking reduction or cessation program.

35. The method of claim 32, wherein the behavior modification program is a program to treat obsessive-compulsive disorder.

36. The method of claim 32, wherein the behavior modification program is a chemical dependency program.

37. The method of claim 32, wherein the behavior modification program is a detoxification program.

38. A method of reducing cravings associated with reducing or eliminating the use of a chemical substance in a human, wherein the method comprises administering to the human an effective amount of a composition comprising an agent to support acetylcholine and an agent to support one or more biogenic amines.

39. The method of claim 38, wherein the chemical substance is selected from the group consisting of nicotine, opioids, methamphetamine, cannabis, and alcohol.

40. A method of reducing cravings associated with reducing or eliminating the use of a chemical substance in a human, the method comprising administering to the human an effective amount of a composition comprising Huperzine A, forskolin, 5-HTP, N-acetyl-cysteine, L-DOPA, and EGCG, or pharmaceutically acceptable salts thereof.

41. The method of claim 40, wherein the chemical substance is selected from the group consisting of nicotine, opioids, methamphetamine, cannabis, and alcohol.

42. A method of supporting appetite control in a human, wherein the method comprises administering to the human an effective amount of a composition comprising an agent to support acetylcholine and an agent to support one or more biogenic amines.

43. The method of claim 42, wherein the human is reducing or eliminating nicotine intake.

44. The method of claim 42, wherein the human is attempting to reduce or eliminate nicotine intake.

45. The method of claim 42, wherein the human is reducing or eliminating smoking.

46. The method of claim 42, wherein the human is attempting to reduce or eliminate smoking.

47. A method of supporting appetite control in a human, wherein the method comprises administering to the human an effective amount of a composition comprising Huperzine A, forskolin, 5-HTP, N-acetyl-cysteine, L-DOPA, and EGCG, or pharmaceutically acceptable salts thereof.

48. The method of claim 47, wherein the human is reducing or eliminating nicotine intake.

49. The method of claim 47, wherein the human is attempting to reduce or eliminate nicotine intake.

50. The method of claim 47, wherein the human is reducing or eliminating smoking.

51. The method of claim 47, wherein the human is attempting to reduce or eliminate smoking

52. A method of treating appetite disturbance associated with initiating a smoking reduction or cessation program in a human, the method comprising administering to the human an effective amount of a composition comprising Huperzine A, forskolin, 5-HTP, N-acetyl-cysteine, L-DOPA, and EGCG, or pharmaceutically acceptable salts thereof.

53. A method of preventing weight gain associated with reducing and/or eliminating nicotine intake in a human, the method comprising administering to the human an effective amount of a composition comprising an agent to support acetylcholine and an agent to support one or more biogenic amines.

54. A method of preventing weight gain associated with reducing and/or eliminating nicotine intake in a human, the method comprising administering to the human an effective amount of a composition comprising Huperzine A, forskolin, 5-HTP, N-acetyl-cysteine, L-DOPA, and EGCG, or pharmaceutically acceptable salts thereof.

55. A method of preventing or reducing weight gain associated with smoking reduction or cessation in a human, the method comprising administering to the human an effective amount of a composition comprising an agent to support acetylcholine and an agent to support one or more biogenic amines.

56. A method of preventing or reducing weight gain associated with smoking reduction or cessation in a human, the method comprising administering to the human an effective amount of a composition comprising Huperzine A, forskolin, 5-HTP, N-acetyl-cysteine, L-DOPA, and EGCG or pharmaceutically acceptable salts thereof.

57. A method of treating appetite disturbance associated with reducing and/or eliminating nicotine intake in a human, the method comprising administering to the human an effective amount a composition comprising an agent to support acetylcholine and an agent to support one or more biogenic amines.

58. A method of treating appetite disturbance associated with reducing and/or eliminating nicotine intake in a human, the method comprising administering to the human an effective amount of a composition comprising Huperzine A, forskolin, 5-HTP, N-acetyl-cysteine, L-DOPA, and EGCG, or pharmaceutically acceptable salts thereof.

59. A method of treating appetite disturbance associated with initiating a smoking reduction or cessation program in a human, the method comprising administering to the human an effective amount of a composition comprising an agent to support acetylcholine function and an agent to support one or more biogenic amines.

60. The method of claim 52, wherein the composition supports healthy efforts to lose weight.

61. The method of claim 52, wherein the composition maintains efficient metabolism in the body.

62. The method of claim 52, wherein the composition supports a balanced appetite.

63. The method of claim 52, wherein the composition facilitates weight loss or weight maintenance.

64. The method of claim 52, wherein the composition reduces Body Mass Index (BMI) of the human.

65. The method of claim 52, wherein the composition enhances a feeling of satiety in the human.

66. The method of claim 52, wherein the composition reduces cravings for a chemical substance.

67. The method of claim 52, wherein the composition supports efforts to reduce or eliminate addictive behavior.

68. The method of claim 52, wherein the composition supports efforts to reduce or eliminate impulsive behavior or reduce or eliminate obsessive behavior.

69. The composition of claim 1, wherein the agent to support one or more biogenic amines can is selected from the group consisting of monoamine reuptake inhibitor (MRIs), selective serotonin reuptake inhibitors (SSRIs), serotonin-norepinephrine reuptake inhibitors (SNRIs), norepinephrine reuptake inhibitors (NRIs), norepinephrine-dopamine reuptake inhibitors (NDRIs), monoamine releasing agents (MRAs), norepinephrine-dopamine releasing agents (NDRAs), tricyclic antidepressant (TCAs), tetracyclic antidepressants (TeCAs), monoamine oxidase inhibitors (MAOIs), 5-HT1A Receptor Agonists, 5-HT2 Receptor Antagonists, Selective Serotonin Reuptake Enhancers (SSREs), sigma receptor agonists, and an anticonvulsant.
